(19) United States
(12) Patent Application Publication
(10) Pub. No.: US 2021/0104174 A1
(43) Pub. Date: Apr. 8, 2021

(54) VOICE ASSISTANT SPEECH LANGUAGE PATHOLOGIST (VA SLP), SYSTEMS AND METHODS

(71) Applicant: NINISPEECH LTD., Haifa (IL)

(72) Inventor: Yoav MEDAN, Haifa (IL)

(21) Appl. No.: 17/060,595

(22) Filed: Oct. 1, 2020

Related U.S. Application Data
(60) Provisional application No. 62/909,865, filed on Oct. 3, 2019.

(51) Int. Cl. G09B 19/04; G10L 25/60; G10L 25/66; A61B 5/00

(52) U.S. Cl. CPC G09B 19/04; G10L 25/60; G10L 25/66; A61B 2505/09; A61B 5/4082; A61B 5/4088; A61B 5/4803

(57) ABSTRACT

There is provided herein a method and system for assisting speech/language therapy practice utilizing a voice interactive artificial intelligence-powered virtual assistant system.

### CROSS REFERENCE TO RELATED APPLICATIONS

This application claims the benefit of priority of U.S. Provisional Application No. 62/909,865 filed on Oct. 3, 2019. The contents of the above applications are all incorporated by reference as if fully set forth herein in their entirety.

### TECHNICAL FIELD

The present disclosure relates generally to systems and methods for utilizing voice assistant for interactive speech/spoken language therapy and practice.

### BACKGROUND

Current speech and spoken language therapy practice is currently based on periodic (e.g. weekly) face-to-face meeting of a speech language pathologist (SLP) with a person (such as, a student, patient, trainee) in a need of speech language therapy and/or practice in order to improve his/her speech skills such as articulation, stutter, language expression, etc. In between such sessions, the SLP typically prescribes some exercises for self-practice, in order to acquire and master the skill, according to some treatment protocol/process.

In practice, people do not tend to adhere to the practice prescription and therefore do not develop or regain the skill as expected. Adherence is known to be a major problem in many therapies, including medical ones.

There is thus a need in the art for systems and methods for encouraging adherence to the prescribed speech therapy practice protocol.

### SUMMARY

Aspects of the disclosure, according to some embodiments thereof, relate to systems and methods for assisting speech/language therapy practice utilizing a voice interactive artificial intelligence-powered virtual assistant system.

There are provided herein, in accordance with some embodiments, a voice assistant speech language pathologist (VA SLP) based systems and methods that utilize VAs as a practice assistance for encouraging and enforcing adherence to the practice prescription. The VAs utilized may be customized/tailor made according to some embodiments of this disclosure or may be an off-the-shelf product, such as but not limited to, Alexa, Siri, Bixby or Google Assistant. Advantageously, the VA engages the trainee in playful activities in order to turn a rather boring activity into a fun and educating experience. This is done, in accordance with some embodiments, by a set of tailored/personalized games in which the VA challenges the trainee and vice versa in an interactive dialogue. The dialogue is continuously monitored (recorded) for extracting Speech Language Qualities (SLQs) and/or attributes that serve as “biomarkers” for gauging and quantifying the quality of speech production and therapy progress according to predefined goals and norms. Such goals and norms may be tailored/personalized to the user's (trainee's) speech/language pathology.

While VAs today offer “interactive” games to general audience, including kids, they are limited to a very short interactions (typically YES/NO answers from the user), which do not serve the purpose of speech therapy. Typically, the VA is talking most of the time, leaving the player passive as far as speaking is concerned.

There is this thus provided herein, in accordance with some embodiments, a VA based gaming system/method, which reverse the roles and put the burden of speaking on the trainee, challenging the VA in order to encourage practice.

There is this provided herein, in accordance with some embodiments, a voice assistant speech language pathologist (VA SLP) based method for assisting speech language therapy practice, the method includes utilizing a voice interactive artificial intelligence-powered virtual assistant system, initiating conversation with a user, wherein initiating conversation with the user is triggered in response to the user's command or triggered by the virtual assistant system, wherein initiating conversation with a user includes: identifying the user and/or uploading a personal speech therapy practice protocol personalized to the user's speech/lingual pathology; based on the personalized practice protocol, requesting the user to perform a task which includes saying one or more words associated with the user's speech/lingual pathology; if the user's speech is determined to be at or above a threshold, rewarding the user with a positive game feature. Optionally, if the user's speech is determined to be below the threshold, the virtual assistant system penalizes the user with a negative game feature or a lack of a positive game feature.

There is further provided herein an interactive artificial intelligence-powered virtual voice assistant speech language pathologist (VA SLP) system for assisting speech language therapy practice, the system including: one or more processors configured to: initiate conversation with a user, wherein initiating conversation with the user is triggered in response to the user's command or triggered by the virtual assistant system, wherein initiating conversation with a user includes: identifying the user and/or uploading a personal speech therapy practice protocol personalized to the user's speech/lingual pathology; trigger, based on the personalized practice protocol, a request to the user to perform a task which includes saying one or more words associated with the user's speech/lingual pathology; determine if the user's speech is at, above or below a threshold, wherein if the user's speech is determined to be at or above a threshold, the processor is configured to reward the user with a positive game feature. The system may further include a remote server, a projector configured to project visual images and/or video clips, a game interface, a monitor, a display unit, an independent mobile device, a microphone, a speaker, a recorder or any combination thereof. Each possibility is a separate embodiment.

The step of requesting the user to say one or more words associated with the user's speech/lingual pathology may include: providing to the user a set of words and requesting the user to repeat them one or more times, providing a user a set of words and requesting the user to re-order them to form a meaningful sentence, providing to the user a set of words and requesting the user to repeat them, playing a sound and asking the user what object/subject produces such sound, describing an object and asking the user to name it, naming and object and asking the user to describe it, projecting a visual image and/or video clips and asking the user to name/describe it or any combination thereof. Each possibility is a separate embodiment.

The step of determining if the speech is at or above a threshold may include analyzing the user's speech quality. Analyzing the user's speech quality may include extracting Speech Language Qualities (SLQs) and/or attributes that serve as “biomarkers” for gauging and/or quantifying the quality of speech production and/or therapy progress according to predefined goals and norms. Such goals and norms may be tailored/personalized to the user's (trainee's) speech/language pathology.

Analyzing the user's speech quality may be performed locally, at a remote server or partially locally and partially at a remote server.

The speech quality may be at least partially determined by the level of similarity between the user's speech and an expected speech.

The level of similarity between the user's speech and the expected speech may be determined based on a number of words which were as expected, a use of synonyms or homonyms, use of words from the same category or any combination thereof. Each possibility is a separate embodiment.

Analyzing the user's speech quality may include determining, evaluating and/or measuring reaction time, number of attempts, order of words, stuttering, omission of words, mispronunciation of words/syllables, length of response time, rate of speech, “swallowing” of words, ratio between mispronounced and correctly pronounced words, speech fluency, use of correct word types, grammar correctness, use of key words (i.e., given a certain prompt by the VA, the user is expected to say certain words, reflecting the richness of their vocabulary), number of correct attempts, length of utterance, pitch of speech, intensity of speech or any combination thereof. Each possibility is a separate embodiment.

It is noted that according to some embodiments, that the content of the speech therapy practice protocol (which may include tasks, games, etc.) is personalized to the user's speech/lingual pathology. The content varies between different users having different pathologies. According to additional or alternative embodiments, each user (e.g., student) may have a different game experience, even for the same content (e.g., game story). For example, if the user has a problem with the pronunciation of the letter “s”, the protocol content may include tasks, which require the sure to say word(s)/sentence(s) with the letter “s”. If the user mixes between “r” and “g”, the content will involve tasks/games which requires using word(s)/sentence(s) with the letters “r” and “g”.

According to additional or alternative embodiments, the content may be dynamic and may vary between users and between practices of the same users. The content may be determined, changed and/or adjusted by the SLP.

The user's speech/lingual pathology may be related to speech/language behavioral, developmental, rehabilitation and/or degenerative related conditions/diseases. The conditions/diseases may be selected from a group consisting of aphasia, Parkinson, Alzheimer's, ALS, LISP speech disorder and stuttering.

According to some embodiments, the user identification may be achieved by recognizing the user's voice, by obtaining a predetermined voice command from the user, by a predefined code/PIN, by a command provided by an independent device (e.g. a mobile device by a text massage) or by any combination thereof.

According to some embodiments, if the user's speech is determined to be at or above the threshold a predetermined number of times, the method further includes a step of increasing (/the processor is further configured to increase) a level of difficulty of a next task presented to the user. If the user's speech is determined to be below the threshold a predetermined number of times, the method further included a step of decreasing (the processor is further configured to decrease) a level of difficulty of a next task presented to the user.

According to some embodiments, initiating conversation with the user may be triggered in response to the user's command, for example but not limited to, voice command. According to alternative embodiments, initiating conversation with the user may be triggered by the virtual assistant system.

According to some embodiments, the voice interactive artificial intelligence-powered virtual assistant system may include Alexa or Google Assistant Siri or Bixby. Each possibility is a separate embodiment.

According to some embodiments, the personalized speech therapy practice protocol includes content, which varies between different users having different speech/lingual pathologies.

According to some embodiments, the personalized speech therapy practice protocol includes content, which provides different game experience for users having different speech/lingual pathologies.

According to some embodiments, the term “user” may refer to a subject, client, student, patient, trainee or any other user.

According to some embodiments, the term “saying” may include speaking, talking, pronouncing, articulating, enunciating, expressing, verbalizing and/or voicing.

Certain embodiments of the present disclosure may include some, all, or none of the above advantages. One or more other technical advantages may be readily apparent to those skilled in the art from the FIGURES, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying FIGURES. The description, together with the FIGURES, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

### In the FIGURES:

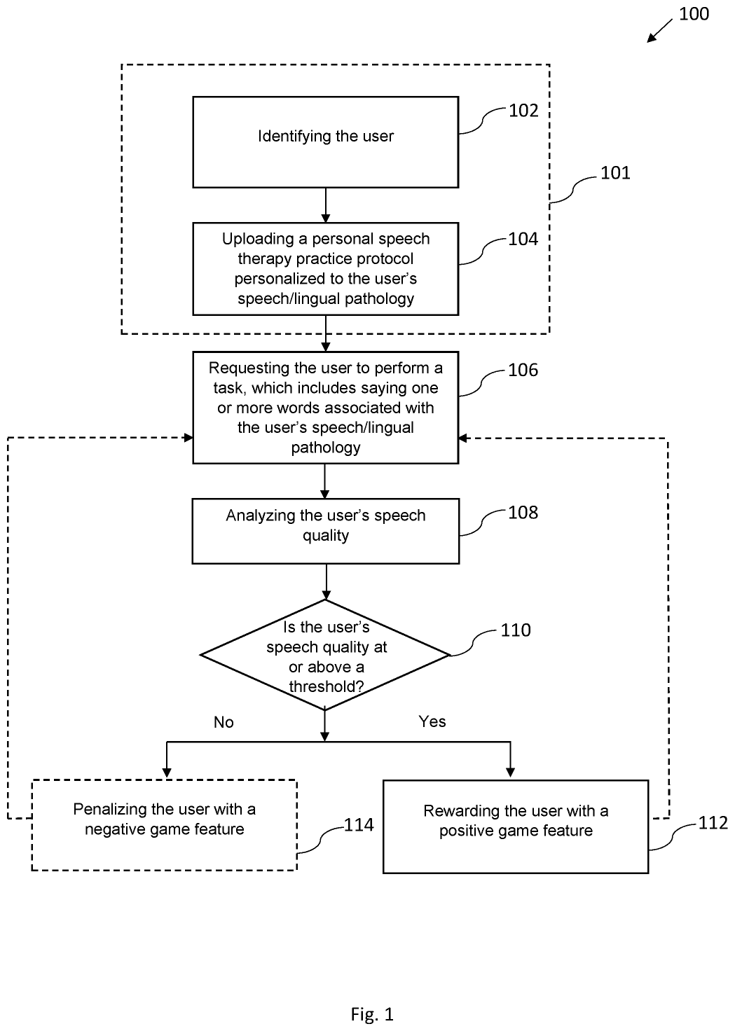

FIG. 1 schematically depicts a flowchart of the method for assisting speech language therapy practice, according to some exemplary embodiments.

### DETAILED DESCRIPTION

The principles, uses and implementations of the teachings herein may be better understood with reference to the accompanying description and FIGURES. Upon perusal of the description and FIGURES present herein, one skilled in the art will be able to implement the teachings herein without undue effort or experimentation. In the FIGURES, same reference numerals refer to same parts throughout.

In the description and claims of the application, the words “include” and “have”, and forms thereof, are not limited to members in a list with which the words may be associated.

Reference is now made to FIG. 1 schematically depicts a flowchart 100 of the method for assisting speech language therapy practice by utilizing a voice interactive artificial intelligence-powered virtual assistant system. The method includes initiating conversation with a user (step 101). Step 101 includes identifying the user (step 102) and uploading a personal speech therapy practice protocol personalized to the user's speech/lingual pathology (step 104). It is noted that in some cases, in accordance with some embodiments, the system may already be assigned to only one user, in which case the step of identifying the user (102) can be avoided. In any case, once the user is identified, the system uploads a personal speech therapy practice protocol, which is personalized to the user's specific speech/lingual pathology (step 104). Moreover, this step of uploading the personal speech therapy practice protocol may not only be personalized to the user's speech/lingual pathology but may also be adapted to the stage of the user in the practice protocol. For example, if the user is only beginning their practice, the system may upload a relatively easy protocol, which involved relatively simple tasks. If the user is already at an advanced level, the system may upload a more difficult protocol, which involved relatively complex tasks. Once the personal practice protocol was uploaded, the user receives from the system requests to perform a task, which includes saying one or more words associated with their specific speech/lingual pathology (step 106). For example, if the user has a problem with the pronunciation of the letter “s”, the protocol may include tasks which require the sure to say word(s)/sentence(s) with the letter “s”. If the user has difficulties with grammar, the tasks will involve tasks that relate to the user's grammar problems. If the user is struggling with stuttering, the system may provide a task that will challenge the speech fluency, etc.

The user's speech is recorded, and the system analyzes the user's vocal response to the tasks and will determine the user's speech quality (step 108). It is noted that, according to some embodiments, the speech analysis may be performed in a remote server. According to other embodiments, the speech analysis may be performed locally, for example in a processor of the voice interactive artificial intelligence-powered virtual assistant. According to some embodiments, the speech analysis may be partially performed in a remote server and partially performed locally, for example in a processor of the voice interactive artificial intelligence-powered virtual assistant. According to some embodiments, the speech may be assigned a score. The score represents the speech quality. The speech quality may be evaluated based on various parameters, such as but not limited to, reaction time, number of attempts, order of words, stuttering, omission of words, mispronunciation of words/syllables, length of response time, rate of speech, “swallowing” of words, ratio between mispronounced and correctly pronounced words, speech fluency, use of correct word types, grammar correctness, use of key words, number of correct attempts, length of utterance, pitch of speech, intensity of speech or any combination thereof. Each possibility is a separate embodiment. The speech quality may also be evaluated based on the level of similarity between the user's speech and the expected speech as determined, for example, based on a number of words which were as expected, a use of synonyms or homonyms, use of words from the same category or any combination thereof. Each possibility is a separate embodiment.

Once the speech quality was evaluated, for example, assigned a score, the system compares the user's speech quality to a predetermined threshold (step 110). If the user's speech quality is above/at the threshold, the system rewards the user with a positive game feature (step 112). Optionally, if the user's speech quality is below the threshold, the system may penalize the user with a negative game feature (step 114). Such reward system underlines the different game interactions, as disclosed herein. This unique approach may for example include using an Avatar, a living organism (such as a plant, pet, baby, etc.) who is nourished by the reward(s) obtain during practice. Practice adherence and success will lead to a prosperity of the Avatar ecosystem. On the other hand, failure to practice and/or to make progress will lead to it diminish. The users are expected to care about their Avatars (such as, but not limited to, Tamaguchi and Furby) and would not want to let them down. Advantageously, this will encourage users to adhere to their speech/language therapy practice protocol and to make a progress in their training.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

What is claimed is:

1. A voice assistant speech language pathologist (VA SLP) based method for assisting speech language therapy practice, the method comprising:
utilizing a voice interactive artificial intelligence-powered virtual assistant system,
initiating conversation with a user, wherein initiating conversation with the user is triggered in response to the user's command or triggered by the virtual assistant system, wherein initiating conversation with a user comprises: identifying the user and/or uploading a personal speech therapy practice protocol personalized to the user's speech/lingual pathology;
based on the personalized practice protocol, requesting the user to perform a task which comprises saying one or more words associated with the user's speech/lingual pathology;
if the user's speech is determined to be at or above a threshold, rewarding the user with a positive game feature.

2. The method of claim 1, wherein if the user's speech is determined to be below the threshold, the virtual assistant system penalizes the user with a negative game feature or a lack of a positive game feature.

3. The method of claim 1, wherein the step of requesting the user to say one or more words associated with the user's speech/lingual pathology comprises: providing to the user a set of words and requesting the user to repeat them one or more times, providing a user a set of words and requesting the user to re-order them to form a meaningful sentence, providing to the user a set of words and requesting the user to repeat them, playing a sound and asking the user what object/subject produces such sound, describing an object and asking the user to name it, naming and object and asking the user to describe it, projecting a visual image and/or video clips and asking the user to name/describe it or any combination thereof.

4. The method of claim 1, wherein the step of determining if the speech is at or above a threshold comprises analyzing the user's speech quality.

5. The method of claim 4, wherein analyzing the user's speech quality is performed locally, at a remote server or partially locally and partially at a remote server.

6. The method of claim 4, wherein the speech quality is at least partially determined by the level of similarity between the user's speech and an expected speech.

7. The method of claim 6, wherein the level of similarity between the user's speech and the expected speech is determined based on a number of words which were as expected, a use of synonyms or homonyms, use of words from the same category or any combination thereof.

8. The method of claim 4, wherein analyzing the user's speech quality comprises determining, evaluating and/or measuring reaction time, number of attempts, order of words, stuttering, omission of words, mispronunciation of words/syllables, length of response time, rate of speech, “swallowing” of words, ratio between mispronounced and correctly pronounced words, speech fluency, use of correct word types, grammar correctness, use of key words, number of correct attempts, length of utterance, pitch of speech, intensity of speech or any combination thereof.

9. The method of claim 1, wherein the user's speech/lingual pathology is related to speech/language behavioral, developmental, rehabilitation and/or degenerative related conditions/diseases.

10. The method of claim 9, wherein the conditions/diseases are selected from a group consisting of aphasia, Parkinson, Alzheimer's, ALS, LISP speech disorder and stuttering.

11. The method of claim 1, wherein the user identification is achieved by recognizing the user's voice, by obtaining a predetermined voice command from the user, by a predefined code/PIN, by a command provided by an independent device or by any combination thereof.

12. The method of claim 1, wherein if the user's speech is determined to be at or above the threshold a predetermined number of times, the method further comprised a step of increasing a level of difficulty of a next task presented to the user.

13. The method of claim 1, wherein if the user's speech is determined to be below the threshold a predetermined number of times, the method further comprised a step of decreasing a level of difficulty of a next task presented to the user.

14. The method of claim 1, wherein initiating conversation with the user is triggered in response to the user's voice command and/or by the virtual assistant system.

15. The method of claim 1, wherein the voice interactive artificial intelligence-powered virtual assistant system is selected from a group consisting of Alexa or Google Assistant Siri and Bixby.

16. The method of claim 1, wherein the personalized speech therapy practice protocol comprises content, which varies between different users having different speech/lingual pathologies and/or wherein the personalized speech therapy practice protocol comprises content, which provides different game experience for users having different speech/lingual pathologies.

17. An interactive artificial intelligence-powered virtual voice assistant speech language pathologist (VA SLP) system for assisting speech language therapy practice, the system comprising: interactive artificial intelligence-powered virtual one or more processors configured to:
initiate conversation with a user, wherein initiating conversation with the user is triggered in response to the user's command or triggered by the virtual assistant system, wherein initiating conversation with a user comprises: identifying the user and/or uploading a personal speech therapy practice protocol personalized to the user's speech/lingual pathology;
trigger, based on the personalized practice protocol, a request to the user to perform a task which comprises saying one or more words associated with the user's speech/lingual pathology;
determine if the user's speech is at, above or below a threshold,
wherein if the user's speech is determined to be at or above a threshold, the processor is configured to reward the user with a positive game feature.

18. The system of claim 19, wherein if the user's speech is determined to be below the threshold, the processor is configured to penalizes the user with a negative game feature or a lack of a positive game feature.

19. The system of claim 19, wherein the step of requesting the user to say one or more words associated with the user's speech/lingual pathology comprises: providing to the user a set of words and requesting the user to repeat them one or more times, providing a user a set of words and requesting the user to re-order them to form a meaningful sentence, providing to the user a set of words and requesting the user to repeat them, playing a sound and asking the user what object/subject produces such sound, describing an object and asking the user to name it, naming and object and asking the user to describe it, projecting a visual image and asking the user to name/describe it or any combination thereof.

20. The system of claim 19, wherein the step of determining if the speech is at or above a threshold comprises analyzing the user's speech quality.
